(19) United States
(12) Patent Application Publication
(10) Pub. No.: US 2015/0164378 A1
(43) Pub. Date: Jun. 18, 2015

(54) PHYSICAL PERFORMANCE ASSESSMENT

(71) Applicant: UNIVERSITY COLLEGE CARDIFF CONSULTANTS LIMITED, Cardiff, South Glamorgan (GB)

(72) Inventors: Trevor Kenneth Baker, Penarth, Vale of Glamorgan (GB); Richard Jasper Day, Penarth (GB); Nicola Phillips, Coity, Bridgend,Vale of Glamorgan (GB)

(73) Assignee: UNIVERSITY COLLEGE CARDIFF CONSULTANTS LIMITED, Cardiff, South Glamorgan (GB)

(21) Appl. No.: 14/395,949

(22) PCT Filed: May 21, 2012
(86) PCT No.: PCT/GB2012/051148

§ 371 Date: Feb. 13, 2015

(30) Foreign Application Priority Data
May 23, 2011 (GB) 1108577.6

(51) Int. Cl. A61B 5/11; A63B 24/00; A61B 5/00

(52) U.S. Cl. CPC A61B 5/1124; A61B 5/1126; A61B 5/7275; A63B 24/0062

(57) ABSTRACT

A method and system (100) adapted to assess performance of at least one physical task. The system includes at least one sensing device (116) configured to output a signal upon activation and an instructing arrangement (112, 114) configured to provide instructions to a subject in relation to performing at least one physical task involving the at least one sensing device. The system further includes a processing device (102) configured to receive data corresponding to signals output by the at least one sensing device. The processing device is further configured to compare the received data with reference data (108) and generate an output based on the comparison representing an assessment of performance of the at least one physical task.

The present invention relates to physical performance assessment.

There is a range of objective measurement tools in the field of functional rehabilitation which can assess and evaluate progress of individuals after injury; for example, muscle strength or range of movement of a joint. The majority of these measures are used within the early and middle stages of rehabilitation. One reason for this is that it is easier to develop and validate measures for an isolated task such as the strength of a specific muscle or the range of movement of a specific joint.

When an individual progresses to late-stage rehabilitation, where the level of functional tasks required becomes more complicated, the ability to measure performance also becomes more complicated; for example, measuring changes of direction whilst running. Furthermore, progression of functional sports rehabilitation involves complex decisions regarding an individual's suitability return to normal activities. This is often described as “back to sport” or “end-stage” rehabilitation. There are very few objective measures or recognised treatment programmes that can quantitatively and reliably measure these types of activity.

Currently, decisions on progression of complexity or return to sport are based on a physiotherapist's subjective assessment of an individual's performance. There are ways of performing objective assessments of performance outcomes, such as timing a sprint task or measuring the accuracy of goal shooting, etc, but very little to quantify the successful completion of more complex tasks needed for most sporting activity. There are several fields, including non-medical fields, other than late-stage rehabilitation where a system capable of providing a more thorough assessment of physical performance of a task is desirable. Examples include sports training and some work-related training, such as military or police roles. Such examples can include a technical rather than biological assessment of the subject's performance for sporting or work-related activities.

Embodiments of the present invention are intended to address at least some of the problems discussed above. Embodiments can provide a system to measure performance of various motor skills and help deliver a structured training programme, such as in rehabilitation or occupational therapy. Embodiments can be particularly helpful for training during late and end-stage functional rehabilitation in a sports context.

According to a first aspect of the present invention there is provided a system adapted to assess performance of at least one physical task, the system including:

at least one sensing device configured to output a signal upon activation;

an instructing arrangement configured to provide instructions to a subject in relation to performing at least one physical task involving the at least one sensing device, and

a processing device configured to receive data corresponding to signals output by the at least one sensing device, the processing device further configured to compare the received data with reference data and generate an output based on the comparison representing an assessment of performance of the at least one physical task.

The processing device may be configured to compare timings of when the signals were output with timings of historical or target sensing device activations in the reference data.

A said sensing device may output a signal indicating contact by, or proximate presence of, the subject. For example, the sensing device may comprise a switch, pressure pad, infra red sensor or a light gate, etc. At least one said sensing device may output a signal representing force exerted by the subject. For example, the sensing device may comprise a piezo-electric sensor membrane. At one of the sensing devices may be spaced apart from other said sensing devices by a distance of at least 0.5 m. The distance may be between 0.5 m and 20 m, e.g. 2-3 m. The sensing devices may be in communication with the processing device by wired or wireless means.

In some embodiments, at least one of the sensing devices may be connected to a physical object that, in use, is carried or manipulated by the subject whilst performing the physical task. For example, the sensing device may be fixed to a ball.

A said sensing device may include a processor that is configured to individually identify the sensing device to another said sensing device and/or the processing device. A processor of the sensing device may communicate with a processor of another said sensing device, e.g. a control message to activate at least one further said sensing device.

The system may further include a video device configured to record at least part of a said physical task. The data recorded by the video device may be processed in order to compare/replay it with the sensing device data.

The instructing arrangement may comprise a visual display device showing a graphical representation of the sensing devices. The visual display device may display textual, pictorial or colour-coded instructions for the subject. Alternatively or additionally, the instructing arrangement may comprise a device configured to output an audible signal.

According to another aspect of the present invention there is provided a method of assessing performance of at least one physical task, the method including:

providing instructions to a subject in relation to performing at least one physical task involving at least one sensing device;

receiving data corresponding to signals output by the at least one sensing device upon activation by the subject during performance of a said physical task;

comparing the received data with reference data, and

generating an output based on the comparison representing an assessment of performance of the physical task by the subject.

A said physical task may involve the subject activating the sensing devices in a particular sequence. For example, the sensing devices may be arranged in pattern (e.g. a zig-zag type arrangement) with a first subset of the sensing devices being located to a left-hand (or right-hand) side of a notional line passing through the pattern and a second subset of the sensing devices being located to a right-hand (or left-hand) side of the notional line. The physical task may involve the subject alternately activating a said sensing device in the first subset and then a said sensing device in the second subset in the particular sequence.

The method may involve processing the data corresponding to the signals output by the sensing devices to generate an output relating to performance of the physical task, the output being selected from a set including:

time taken by the subject to perform the physical task in its entirety;

time taken between the subject activating at least one sensing said device in the first subset and at least one said sensing device in the second subset (representing time taken to transfer between left-hand and right-hand sensing devices), or vice versa;

time taken by subject to progress between a first pair of said sensing devices in the sequence, a second pair of said sensing devices in the sequence, and so on;

approach speed of the subject to the sensing device, and/or

time spent by the subject in contact with at least some of the sensing devices in the sequence.

A said physical task may include the subject moving from one said sensing device to another said sensing device. The physical task may include a further activity in addition to moving from the sensing device to another. For example, the further activity may involve a decision-making task and the method may time/derive time taken in relation to the decision-making.

A physical task may involve the subject directly or indirectly applying physical force to a said sensing device, the sensing device outputting, in use, a signal corresponding to the physical force applied by the subject.

A said physical task can include the subject moving from one said sensing device to another said sensing device in a specific way, e.g. running, jogging or hopping on a specified leg. When the subject is hopping then the method may measure times when the subject is hopping on each leg. Measurements taken or computed by the method can include: time in flight whilst hopping; time on spent on the sensing devices; split times in flight and on the sensing devices; number of contacts per said sensing device; and/or differences between right and left leg/preseason/normal.

According to a further aspect of the present invention there is provided a computer program product comprising a computer readable medium, having thereon computer program code means, when the program code is loaded, to make the computer execute a method substantially as described herein. A device, such as a computing device, configured to execute methods substantially as described herein may also be provided.

Whilst the invention has been described above, it extends to any inventive combination of features set out above or in the following description. Although illustrative embodiments of the invention are described in detail herein with reference to the accompanying drawings, it is to be understood that the invention is not limited to these precise embodiments. As such, many modifications and variations will be apparent to practitioners skilled in the art. Furthermore, it is contemplated that a particular feature described either individually or as part of an embodiment can be combined with other individually described features, or parts of other embodiments, even if the other features and embodiments make no mention of the particular feature. Thus, the invention extends to such specific combinations not already described.

The invention may be performed in various ways, and, by way of example only, embodiments thereof will now be described, reference being made to the accompanying drawings in which:

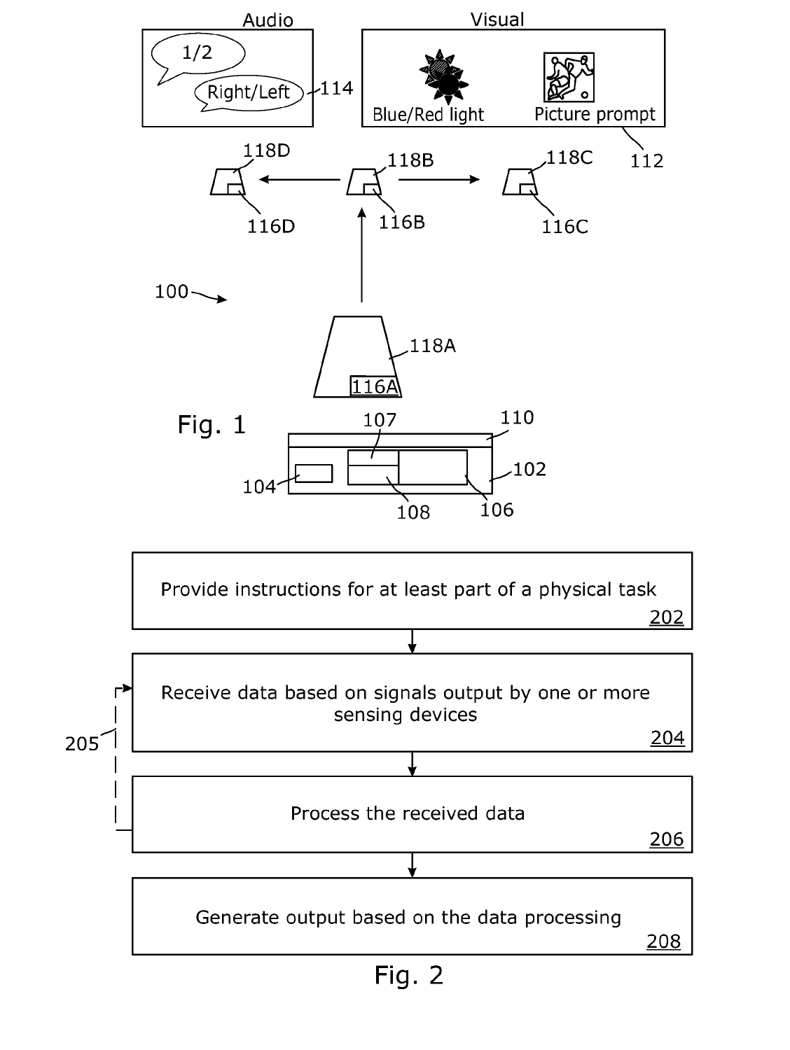

FIG. 1 is a schematic drawing of an example system for assessing performance of physical tasks;

FIG. 2 is a flowchart illustrating example steps performed by the system;

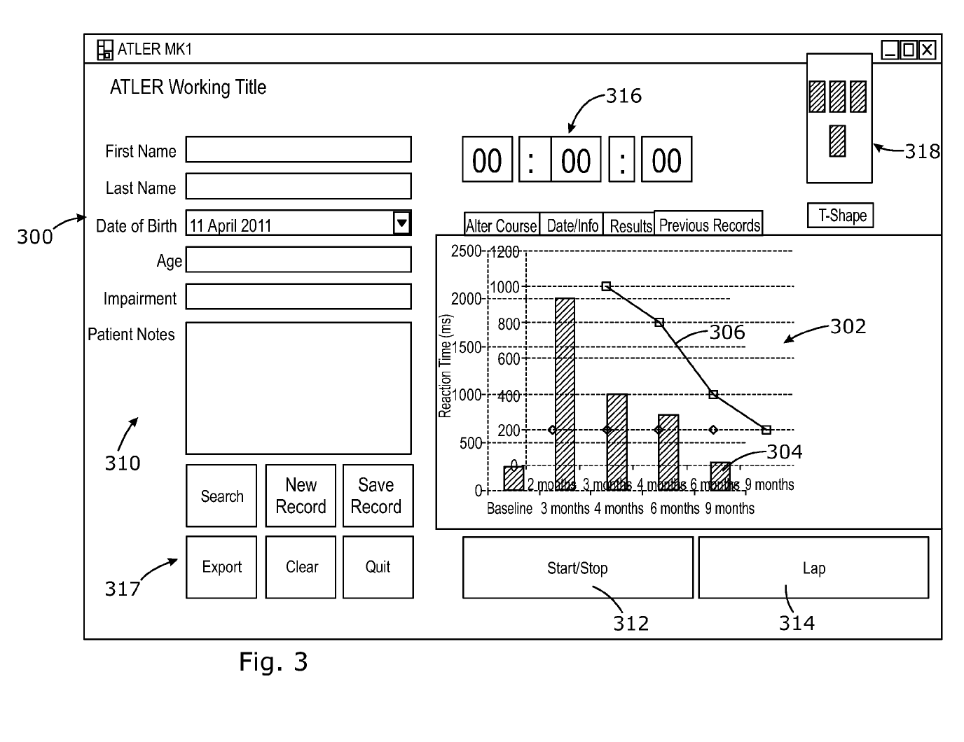

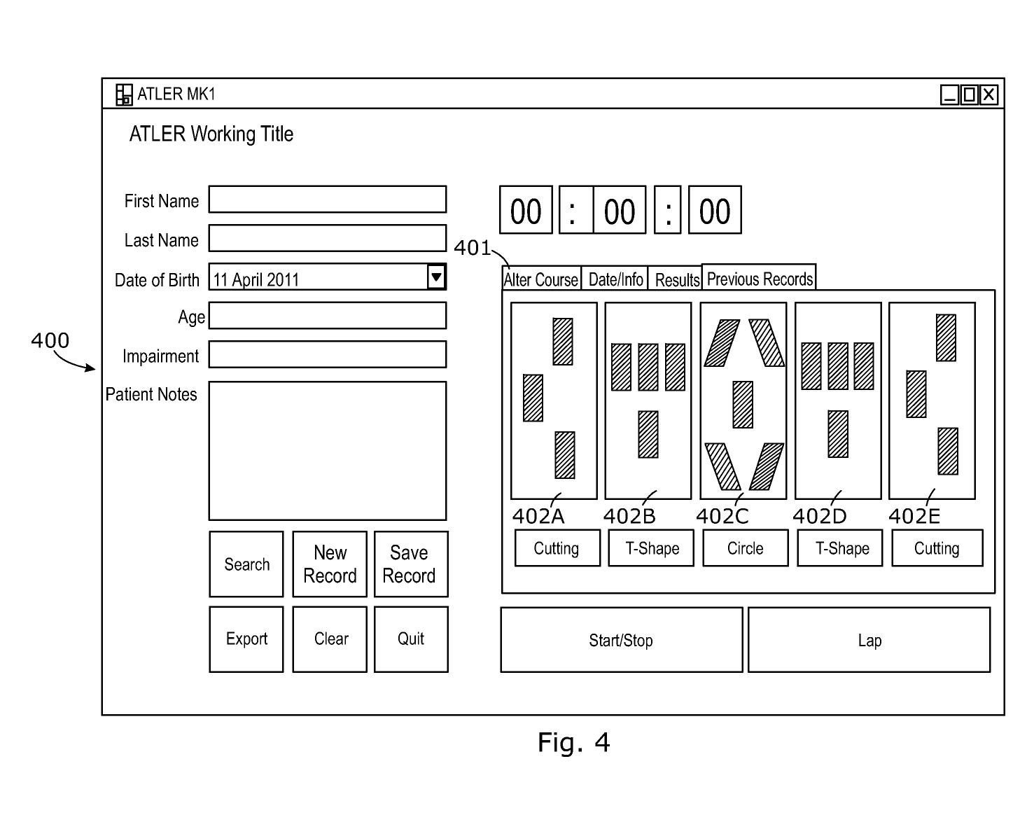

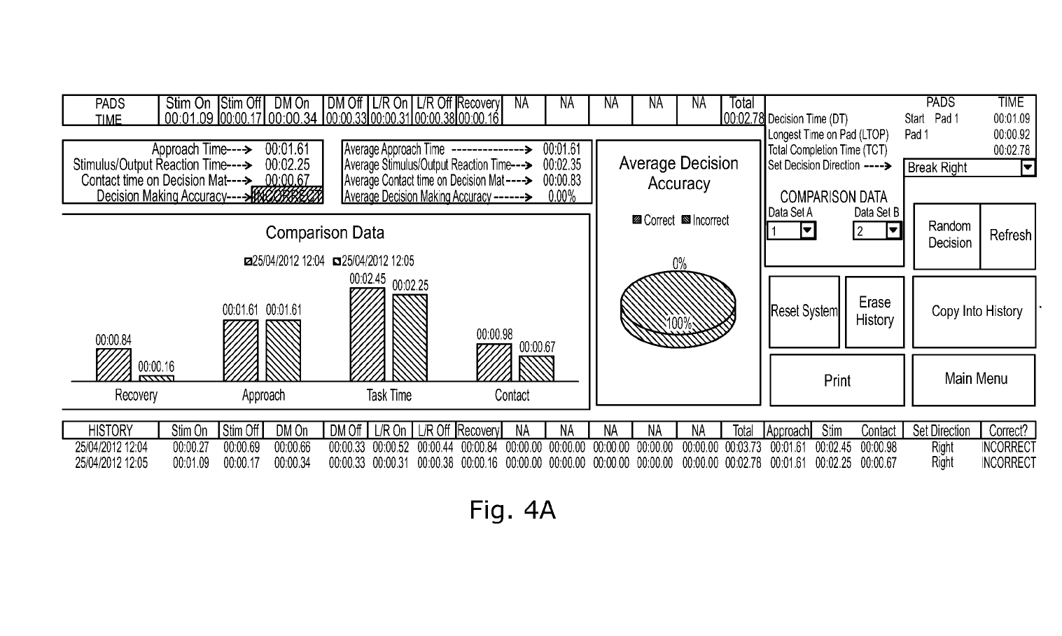

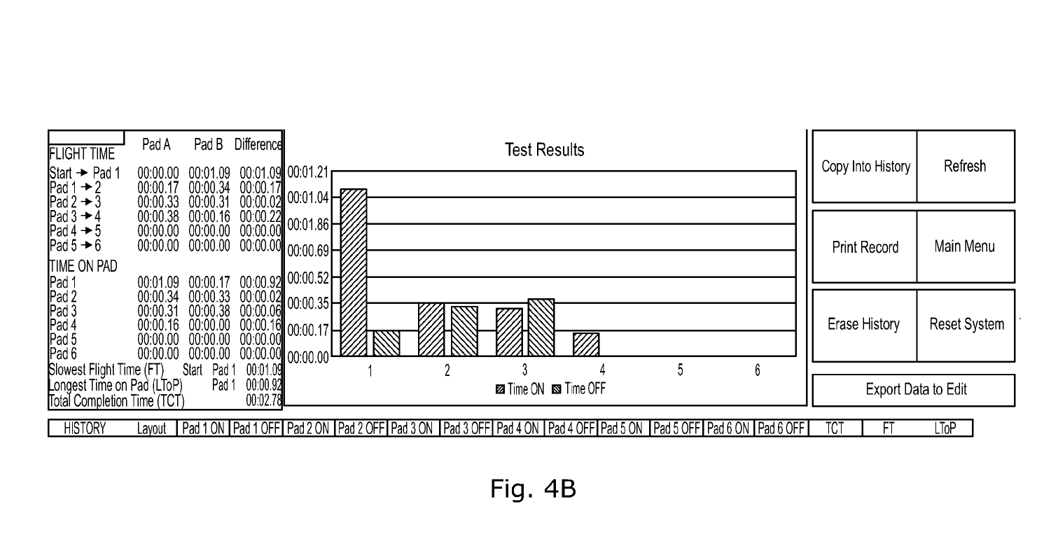

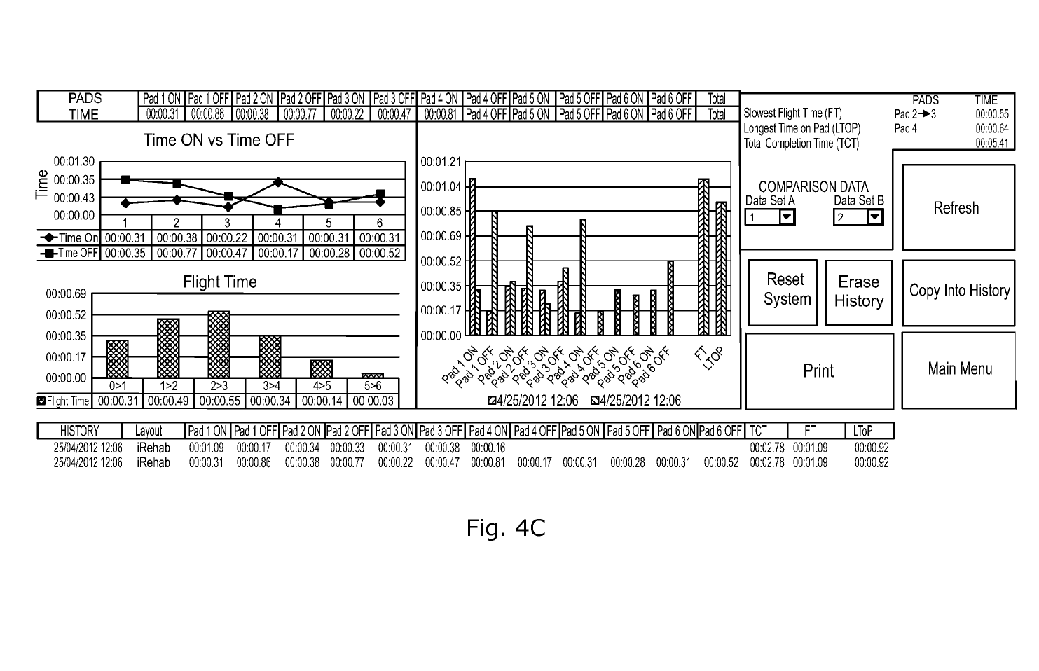

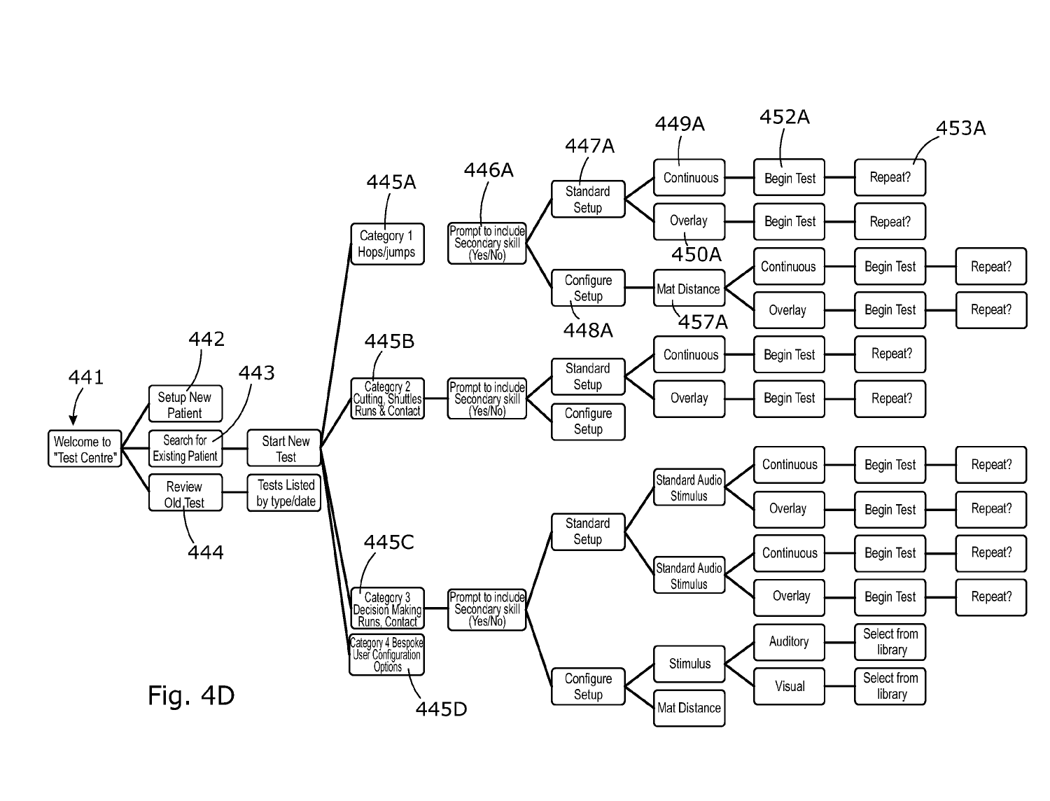

FIGS. 3, 4, 4A, 4B and 4C are example screen displays generated by the example system;

FIG. 4D illustrates schematically options that may be offered to a user of an example system;

FIG. 5 is a schematic illustration of an alternative set up of sensing devices for the system, and

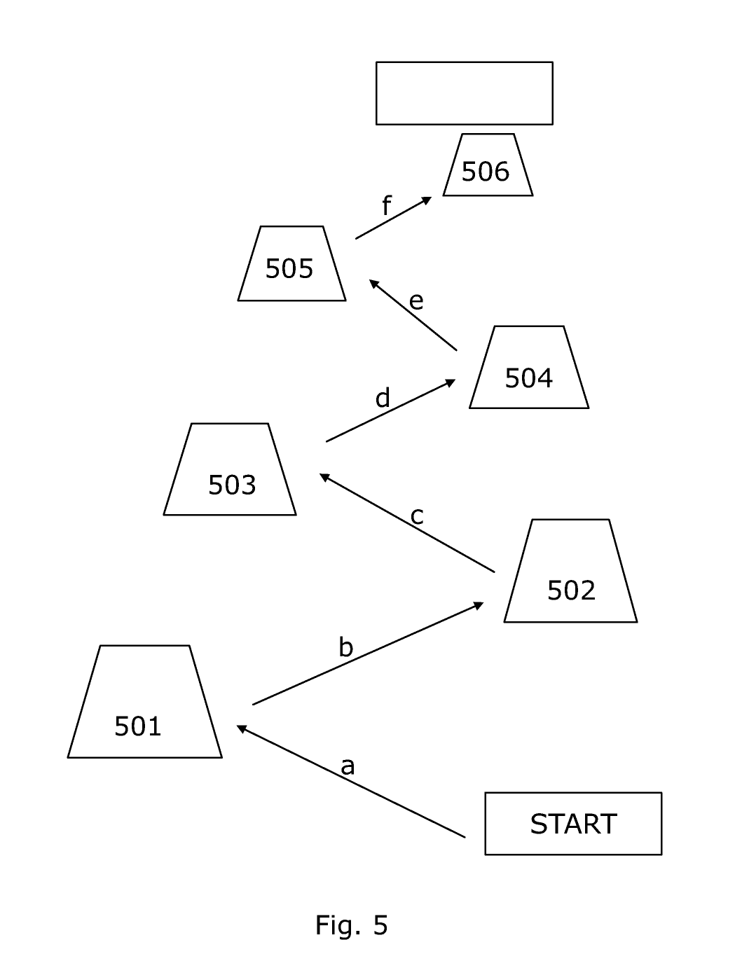

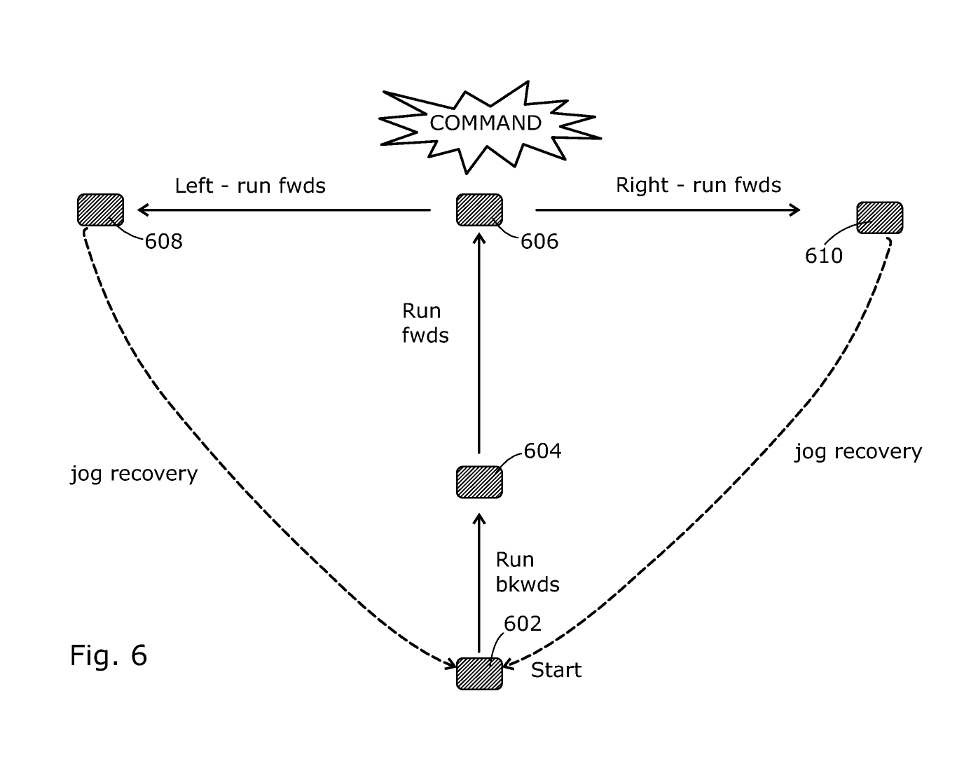

FIGS. 6 to 8 show further example set ups of sensing devices for embodiments of the system.

Referring to FIG. 1, an example system 100 for assessing performance of physical tasks includes a computing device 102 having a processor 104 and memory 106. Other common elements of the computing device, e.g. external storage, are well known and are not shown or described for brevity. The memory 104 includes an application 107 for assessing physical task performance and related data 108.

The computing device 102 includes a communications interface 110 that is able to transfer data to/from remote devices, including a remote display 112 and audio device 114. The system further includes a set of sensing devices 116A-116D. In one embodiment the sensing devices comprise pressure sensitive switches encased in floor mounted pads 118A-118D and are linked to the computing device's interface by means of a computer-controlled switch box 120. It will be appreciated that the number and arrangement of the of sensing devices/pads are exemplary only and many variations are possible. Further, all of the sensing devices need not be of the same type. The pads may include a processor (or at least an RFID device or the like) that allows them to be individually identified by each other and/or the computing device. In some cases, the processors of the pads may communicate with each other; for instance, if one of the pads is activated then it can send a control/activation message to at least one other pad. In another example a pad can re-start a test automatically to measure attenuation rate over time. It will be appreciated that in some embodiments, at least some of the functions performed by the computing device 102 can be implemented by means of hardware executing on one or more of the pads. Further, data could be backed-up or uploaded or storage/processing via a network/cloud. The pads can be arranged so as to allow significant physical activity to take place involving them. In some cases the subject will be required to walk or run between the pads and so there may be a minimum distance of at least 0.5 m between at least one pair of pads/sensing devices and the distance may be up to around 10 m, and in the case of arrangements for use with sprint tests and the like, up to around 20 m.

The system 100 shown in FIG. 1 can be used to test a combination of motor and cognitive skills typical of sports activity. Rehabilitation progression usually involves the addition of multiple tasks and decision-making skills to a functional skill. The skill may involve any combination of direction change in response to a given command, which could be either an auditory or visual in various forms. A secondary skill, such as ball control, increases the complexity of the task and recreates the true “back to sport” level of skill required for participation fitness.

FIG. 2 shows general steps performed by embodiments of the system. At step 202 a person (test subject) who is to be assessed by the system is given an instruction for at least part of a physical task involving one or more of the sensing devices 116. In some embodiments the instruction may be conveyed by the system hardware, e.g. by the remote audio device 114 issuing a verbal or coded audio command, or by means of textual, pictorial or colour-coded means displayed on the remote screen 112. For example, the mats containing the sensing devices may have different colours and the screen may display a colour, thereby instructing the subject to run to the mat having that colour. Alternatively, the screen may display an arrow and the subject should run to the pad in the direction of the arrow. In alternative embodiments, the subject may be given instructions by another arrangement, e.g. reading them from a sheet or being verbally instructed by a supervisor or a user of the system 100.

At step 204 the application 107 waits for data to be received based on signals output by one or more of the sensing devices 116 and records this. The application typically stores data relating to the identity of the sensing device(s) that produced the signal(s) as well as data relating to the timing of the signal, e.g. the time when the signal was received by the computing device which substantially corresponds to the time when the sensing device was activated, indicating when the subject was at a particular location. It will be appreciated that the data can be stored in any suitable format and other types of information can also be stored, e.g. a value representing a force measurement taken by a sensing device. Signals output by a sensing device can include, for example, the approach speed and/or the decision time (e.g. time taken by the subject on and between each sensor).

In some cases (as illustrated by arrow 205), control may return at least once to step 202 and another instruction relating to the physical task is given to the user, followed by recording data from sensors involved in the performance of that instruction at step 204 again.

After the application 107 has received an input indicating that performance of the task has been completed, such as the user activating the final sensing device in a sequence (or step 204 ending in some other way, e.g. timed out, or a user of the computing device indicating that no further input is to be expected, etc) then at step 206 the application processes the recorded data. In general terms, this processing typically involves comparing the recorded timings of sensing devices being activated with reference data. The reference data may be based on one or more previous performance by the subject, or may be data representing, for instance, average timings for performance of the task by a person matching the subject's age/gender profile. Information regarding the subject, such as age, gender, weight, etc, may be entered into/stored by the application.

At step 208 the application 107 generates an output based on the data processing of step 206. It will be appreciated that in alternative embodiments, an output may sometimes additionally be generated upon receiving data at step 204, e.g. to update an onscreen representation of a sensor being activated substantially in real time. The output can take various forms, ranging from a simple “pass/fail” type indication (dependent on whether the subject's performance was worse or matched/better than the reference data) to more complex analysis of the timings and/or associated physical information. For instance, the output can indicate that the force exerted by the subject onto a force sensor is a percentage of an expected value. Such information may be displayed in numerical or graphical form, e.g. a “sliding scale”. Outputs for comparing the subject's performance of tasks over several attempts/time can be produced, e.g. to assess the subject's performance as a result of training, or development with age. The output may be displayed by the computing device 102 and/or stored or transferred to another device for future use.

FIG. 3 shows an example screen display 300 that can be generated by the application 107 at step 208. The display includes a graph 302 showing data relating to a subject's reaction time (the y-axis) over a period of several months of using the system (the x-axis). The graph may be in the form of bars 304 that have different colours representing different aspects of performance. Alternatively or additionally, the graph may be in the form of a line graph comparing the user's recorded performance 306 with baseline/reference performance 308. The display can also include a region 310 for showing personal data relating to the subject, as well as control icons 312, 314 for timing the subject's performance, or for testing. For example, the “Start” control icon 312 may be pressed when the user is told to commence the task by the application user, prior to any sensor being activated. An indication 316 of the time since starting performance of the task may also be displayed. There is also a group 317 of icons for creating, searching, editing and saving/exporting the data.

The display of FIG. 3 also includes an indication 318 of the type of physical task to which the data relates. In the example, the task involves sensing devices fitted in a T-shaped arrangement of floor pads. FIG. 4 shows another screen display 400 produced by the application 107 (by selecting the “Alter course” tab 401) that allows a user to select from a set of different physical tasks 402A-402E. Some embodiments of the system will also require the physical arrangement of the sensing devices to be altered to correspond to the selected arrangement, whereas in embodiments where the sensing devices are part of a configurable matrix of sensing devices, for example, then the software may control which of the devices can be activated for a selected task. FIGS. 4A, 4B and 4C show other data display and processing options that can be produced by embodiments of the system.

FIG. 4D illustrates (menu) options that can be presented to a user in an embodiment of the system. A welcome message 441 can take the user to a main menu that includes options to setup a new patient/subject 442; search for data relating to an existing patient 443 or review an old test 444. For a new patient selected using option 442, the user can be given an option to start a new test. In the example system there are four categories 445A-445D of tests. In the case where the user selects the first test category 445A (mats/jumps) then an option 446A may be offered to the user as to whether or not they want include tests of secondary skills in the test. The user can then select a standard test setup 447A, or a user-configured setup 448A (e.g. the user can setup parameters, such as the maximum distance to be covered by the patient during the test 451A). The user can be allowed to select whether the data from the test is continuous 449A (e.g. added to the patient's existing record) or overlays 450A existing data. The user can then start the test 452A and after it has been executed then the user can be given the option to repeat 453A the test.

A more detailed description of example operations of the system will now be given. In one example the physical task begins with an instruction for the subject to run from pad 118A to 118B of FIG. 1. Contact with the sensor 116B of pad 118B not only measures the time taken to run from pad 118A, but can also act as the trigger for an audio and/or visual prompt. The prompt can be linked to the application 107 and generated as required, depending on the nature and complexity of stimuli needed. The time spent by the subject on pad 118B is measured and provides a reaction time to the stimulus prompt, i.e. an indication of how look it took the subject to decide in which direction to run next. The subject acts on their cognitive decision from the audio/visual prompt and moves to either pad 118C or 118D in response to the command. Contact with the sensing device in pad 118C or 118D finishes the task and completes the time data for analysis.

In other embodiments, objects may be incorporated into the physical tasks. For instance, conductive strips can be attached to equipment such as a ball and can be used to provide signals for assessing performance of a skill. The task may involve the subject also having to catch or kick the ball at the same time as being given commands related to direction. The sensing devices in the floor pads can give information on when contact was made and the sensing device attached to the ball can give information on whether (and when) the ball was caught or kicked. It will be understood that many variations of this are possible, e.g. any suitable type of sensor may be fitted onto any piece(s) of sporting equipment to be used by the subject (e.g. a tennis racquet or the like).

When using the above example systems a subject with a pathology or functional impairment is likely to take longer to respond to a stimulus and may also be more likely to make an incorrect decision or fail the additional secondary task as well as exhibit altered load values.

FIG. 5 shows an arrangement of sensing devices for use by the system in the testing of a rehabilitation skill known as “cutting”. Cutting involves moving through the sequence of pads (fitted with the sensing devices) numbered from 501 to 506 in the direction of the arrows a-f. The task typically involves rapid change of direction, which requires advanced weight transference skills, joint loading, joint rotation and impact as well as, acceleration and deceleration forces of the lower limb. However, in this example cutting is the only task being completed, with no other cognitive or motor tasks involved.

In an alternative example task, called “Cutting hop”, the pads of FIG. 5 are used in a different manner. The instructions given to the subject can be along the lines of: “You have to hop using a designated leg from the start line onto pad one and then hop from pad to consecutive pad”. The aims of the task can include: the individual hops on a designated leg from pad 1 consecutively to pad 5; the individual jumps as high as he/she can from pad to pad; the individual should spend as little time as possible on each pad; the individual should have even times on left and right leg.

In another example task, the system may be configured so that a subject is instructed to run from a starting point to a second point, racing against another individual. The subject may then be instructed to tackle the other individual upon reaching the second point to obtain a ball from them (with the ball or individual having a sensor to assess the timing and/or force of the tackle). The subject may then be instructed to run back to the starting point. Timing data and other information for performance of this task can then be analysed and output by the application. Sensors may also be incorporated into tackle bags or the like, or fitted to surfaces that may be horizontal (floor or ceiling), vertical or angled.

Further examples of tasks are given in the table below:

| | | Improved | Key | |
|---|---|---|---|---|
| | | performance | standardisa- | |
| Test | What it does | indicators | tion factors | Comments |
| Horizontal | Indicator of | Mat contact time | Distance | Distance |
| repeated | plyometric | [decreased contact | between | between |
| hop | ability over a | time indicates | mats needs | mats (ie |
| | series of hops | improvement] | to be | horizontal |
| | | No. contacts per mat | recorded for | hop ability) |
| | | [single contact | each test | is likely to |
| | | indicates better control] | | increase |
| | | Possibly flight versus | | as they get |
| | | contact ratio but will | | stronger - |
| | | need to produce data | | therefore |
| | | to support those claims | | distance |
| | | Timing of whole task | | expected |
| | | Right to left comparison | | to hop has |
| | | | | same |
| | | | | functional |
| | | | | balance |
| | | | | challenge |
| | | | | would |
| | | | | increase -- |
| | | | | this |
| | | | | would |
| | | | | make |
| | | | | comparison |
| | | | | with |
| | | | | earliest |
| | | | | tests |
| | | | | impractical - |
| | | | | so typical |
| | | | | progression |
| | | | | would be |
| | | | | to increase |
| | | | | in stages |
| | | | | and record |
| | | | | increase a- |
| | | | | thus |
| | | | | allowing |
| | | | | limited |
| | | | | comparison |
| | | | | with |
| | | | | earlier |
| | | | | tests as |
| | | | | well as R |
| | | | | vs left |
| Cross over | Indicator of | Mat contact time | Distance | a/a - plus - |
| hop | plyometric | [decreased contact | between | standard- |
| | ability over a | time indicates | mats needs | isation of |
| | series of hops - | improvement] | to be | WHERE |
| | with a more | No. contacts per mat | recorded for | measured |
| | functional lateral | [single contact | each test | from when |
| | challenge | indicates better | | looking at |
| | | control] | | diagonal |
| | | Possibly flight versus | | distances |
| | | contact ratio but will | | |
| | | need to produce data | | |
| | | to support those claims | | |
| | | Timing of whole task | | |
| | | Right to left | | |
| | | comparison | | |
| Cutting | Indicator of | Mat contact time | Distance | a/a |
| | lateral control | [decreased contact | between | |
| | [with plyometric | time indicates | mats needs | |
| | ability if mats | improvement] | to be | |
| | close enough | No. contacts per mat | recorded for | |
| | for no step in | [single contact | each test | |
| | between] | indicates better control] | | |
| | change of | Possibly flight versus | | |
| | direction if | contact ratio - | | |
| | further apart to | increased time here | | |
| | include | means slower run | | |
| | step/stride | times/but if straight | | |
| | | from one mat to | | |
| | | another it works the | | |
| | | same as for hops but | | |
| | | looking at a leap | | |
| | | instead which is | | |
| | | technically easier | | |
| | | than a hop | | |
| | | Timing of whole task | | |
| | | Right to left | | |
| | | comparison | | |
| T shape - | Lateral control | Mat contact time on | Distance | Distance is |
| cutting in | as for cutting | central mat of T | between | likely to |
| response | but with | [decreased contact | mats needs | stay |
| to command | response/reaction | time indicates | to be | standardised |
| | times | improvement] | recorded for | for this |
| | | Time between 1^{st} mat | each test | test as not |
| | | and 2^{nd} gives | | given this |
| | | approach running | | test until |
| | | speed - which will be | | they are |
| | | slower if they are | | doing |
| | | deliberately giving | | similar |
| | | themselves more | | drills in |
| | | time to react to | | rehab and |
| | | stimulus | | it would |
| | | Time between | | therefore be |
| | | contact mat triggering | | standardised |
| | | command and central | | as a test |
| | | mat of T gives | | regardless |
| | | reaction time - and | | of whether |
| | | added to mat contact | | injured or |
| | | time gives total | | not or |
| | | reaction time | | stage in |
| | | Total time from | | rehab |
| | | command to last mat | | |
| | | as run through area | | |
| | | gives total task time - | | |
| | | smaller as they | | |
| | | improve | | |
| Square | Multi-direction | Time from command | Distance | Distance is |
| | control in | to reaching target mat | between | likely to |
| | response to | [decreased contact | mats needs | stay |
| | visual stimulus | time indicates | to be | standardised |
| | (can also be | improvement] | recorded for | for this |
| | auditory but | Time from command | each test - or | test as not |
| | probably more | to returning to centre | size of grid | given this |
| | useful as visual | mat [decreased | with mats on | test until |
| | as more | contact time indicates | periphery | they are |
| | applicable to | improvement] | | doing |
| | racquet sports) | Notification of number | | similar |
| | | of correct and | | drills in |
| | | incorrect decisions | | rehab and |
| | | Choice of 1-4 | | it would |
| | | | | therefore be |
| | | | | standardised |
| | | | | as a test |
| | | | | regardless |
| | | | | of whether |
| | | | | injured or |
| | | | | not or |
| | | | | stage in |
| | | | | rehab |
| | | | | Also it is |
| | | | | likely to |
| | | | | represent |
| | | | | ½ court |
| | | | | size |

Utilizing the hardware and software described above a range of “time” outcome measures can be collected, examples of which include:
Time of overall task.
Time of left to right transference (mean time in direction of arrows a, c and e) compared to right to left transference (mean time in direction of arrows b, d and f).
Attenuation, the timing of progression through the pads, e.g. time from start point to 501, 501 to 502, 502 to 503, 503 to 504, etc.
This provides an indication of the effect of fatigue on change of direction speed and a graphical display comparing the subject's performance of this task on several occasions may be produced.

Time spent in contact with the pads compared to other pads in sequence, for example, either left pads (501, 503, 505) to right pads (502, 504, 506) or from start to 501, 501 to 502, 502 to 503, 503 to 504, etc. Additionally, timing data can be produced to provide information on the subject's performance during left-to-right and right-to-left phases of a task.

FIG. 6 shows an alternative layout of sensing devices 602-610, arranged in a “T” shape. The subject may be instructed to run/jog (in a backwards or forward direction) from the starting sensor 602 to a second sensor 604 and then a further sensor 606. Instructions can then be provided for the subject to run to the upper left-hand 608 or right-hand 610 sensing device. An example task involving this arrangement is called “Decision T”, which involves measurement including time take to change direction by 90° . The instructions for the task can be along the lines of “You have to run from pad 602 towards pad 606. When you touch pad 604, a command will be given. This will instruct you to either turn left or right. When you hear/see this command you have to choose the correct direction and get to that pad (to pad 608 or 610) as quickly as possible”. Thus, the individual runs from pad 602 towards pad 606, during which contact with pad 604 triggers a selected command (sound/light/image). This command instructs the individual which direction to run, i.e. towards pad 608 or pad 610. The aims of this task can include: transfer from pad 602 to pad 606 and the selected pad (608 or 610) in the shortest possible time; spend as little time as possible on pad 606; make the correct decision regarding the new direction of travel. The measures and inference of measures can include: 1) Time from pad 602 to pad 606 [shorter time better performance]; 2) Time from pad 602 to pad 608/610 [shorter time better performance]; 3) Time on pad 606 [Shorter time better performance]; 4) Correct decision [higher % of correct decision better performance]; 5) Differences in time between left or right change of direction [even left/right times=better performance, a difference in time may indicate unilateral stability or confidence in WB issues].

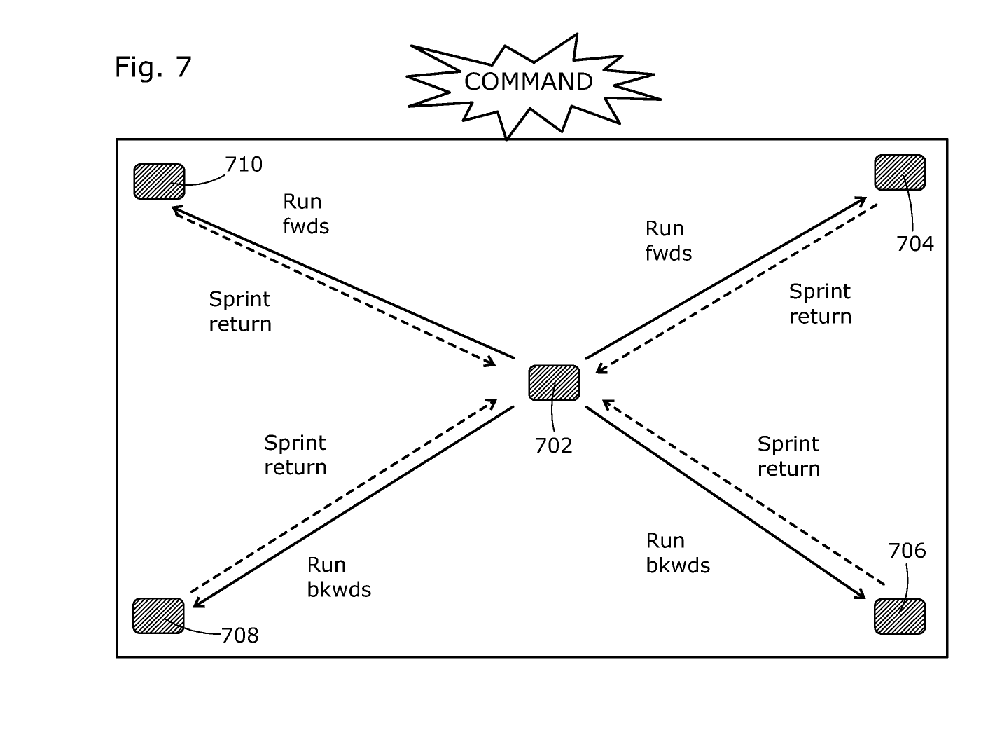

FIG. 7 shows yet another arrangement of sensing devices, including a first sensing device 802 located in the centre of a 2×2 matrix of sensors 804, 806, 808, 810. Again, the subject can be instructed to run/spring/jog in a forwards or backward direction between any combination/series of these sensing devices.

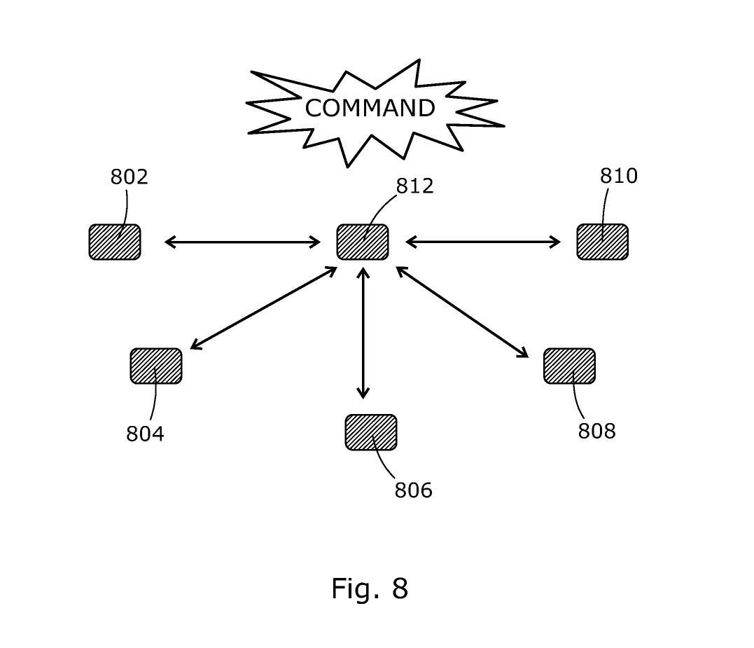

FIG. 8 shows another arrangement of sensors where a set of five sensors 802, 804, 806, 808, 810 are arranged in a semi-circular manner, with a further sensor 812 located in the centre of the diametrically opposed sensors 802, 810.

Arrangements of sensing devices like the ones shown in the Figures can be used to provide running drills for various sports. The arrangement of FIG. 6 can be particularly useful for field sports (e.g. football, rugby, field hockey, Lacrosse, etc). The arrangement of FIG. 7 can be particularly useful for racquet sport (tennis, squash, badminton, etc). The arrangement of FIG. 8 can be useful for various sport, particularly ones involving short distance requiring forwards/backwards/sideways movement, or rapid control short distances for marking/defensive movement (e.g. basketball, tennis, netball).

Another example task, called “Straight hop”, involves a set of sensing devices (e.g. 5) arranged in a straight line. The instructions given to the subject can be along the lines of: “You have to hop using a designated leg from the start line onto pad one and then hop from pad to consecutive pad”. Aims of the task can include: the individual hops on a designated leg from the first pad in the set consecutively to the last pad; the individual to jump as high as he/she can from pad to pad; the individual should spend as little time as possible on each pad; the individual should spend even times on left and right leg. The measures and inference of measures can include: 1) time in flight [longer time in flight; 2) time on pads [shorter time=better performance]; 3) split times in flight and on pads [even split times=better performance]; 4) number of touches per pad [one touch per pad=better performance]; 5) differences between right and left leg/preseason/normal.

It will be appreciated that such timing measurements can be made for other tasks/arrangements of sensing devices. For example, the subject may be asked to perform the same task under different conditions, e.g. whilst wearing an article, or after ingesting a product, that is claimed to enhance performance. The results output by the application may be used to help verify or disprove such claims. Other embodiments of the system can include the ability to measure a load applied to a sensing device as well as a time variable. The pads can include an inbuilt switch to activate timing measures as well as a piezoelectric sensor membrane, which can measure the specific load applied to the pad. This can enable more advanced interpretation of the individual's functional ability through individual loading measures as well as time/load ratios. In other embodiments, the system may further include a video device, such as a webcam, that can record at least part of the session. The video data may be processed in order to compare/replay it with the sensor device data.

Embodiments of the present system can enable objective and interpretable data to be collected and potentially referenced to normative values for recreational level or to pre-injury values for high performance sport, as well as for many other types of physical tasks. Embodiments may be used to assess the mobility of homebound patients, e.g. people with Alzheimer's or other dehabiliatating conditions. The hardware also demonstrates huge flexibility for the physiotherapist or other user to format the task specific to their sporting/functional requirements. Furthermore, the system can also be easily adapted to other skills, for example, the sensing devices can be easily integrated into tackle pads in a rugby setting to measure the time performance of a rugby player running through a predetermined sequence of contacts. The hardware and software programming capability also exists to allow for complete wireless (e.g. WiFi) functionality which would allow sensing devices to be placed in a variety of units other than floor pads; for example, cones using light beam/laser switches.

1. A system (100) adapted to assess performance of at least one physical task, the system including:
at least one sensing device (116) configured to output a signal upon activation;
an instructing arrangement (112, 114) configured to provide instructions to a subject in relation to performing at least one physical task involving the at least one sensing device, and
a processing device (102) configured to receive data corresponding to signals output by the at least one sensing device, the processing device further configured to compare the received data with reference data (108) and generate an output based on the comparison representing an assessment of performance of the at least one physical task.

2. A system according to claim 1 , wherein the processing device (102) is configured to compare timings of when the signals were output by sensing devices (116) with timings of historical or target sensing device activations in the reference data (108).

3. A system according to claim 2, wherein a said sensing device (116) outputs a said signal indicating contact by the subject.

4. A system according to claim 2, wherein a said sensing device (116) outputs a said signal indicating proximate presence of the subject.

5. A system according to claim 3, wherein the sensing device (116) comprises a switch, pressure pad, infra red sensor or a light gate.

6. A system according to claim 1, wherein at least one said sensing device (116) outputs a signal representing force exerted by the subject.

7. A system according to claim 6, wherein the sensing device (116) comprises a piezoelectric sensor membrane.

8. A system according to claim 1, wherein at one of the sensing devices (116A) is spaced apart from another said sensing device (116B) by a distance of at least 0.5 m.

9. A system according to claim 8, wherein the distance is between 0.5 m and 20 m.

10. A system according to claim 1, wherein a said sensing device (116) is connected to a physical object that, in use, is carried or manipulated by the subject whilst performing the physical task.

11. A system according to claim 10, wherein the sensing device (116) is fixed to a ball.

12. A system according to claim 1, wherein at least one said sensing device (116) includes a processor that is configured to individually identify the sensing device to another said sensing device and/or the processing device (102).

13. A system according to claim 12, wherein the processor of the sensing device (116) communicates with a processor of another said sensing device, e.g. sends a control message to activate the other said sensing device.

14. A system according to claim 1, further including a video device configured to record at least part of a said physical task.

15. A system according to claim 15, wherein the data recorded by the video device is processed in order to compare/replay it with the sensing device data.

16. A system according to claim 1, wherein the instructing arrangement comprises a visual display device (112) configured to show a graphical representation of at least one of the sensing devices (116).

17. A method of assessing performance of at least one physical task, the method including:
providing instructions (202) to a subject in relation to performing at least one physical task involving at least one sensing device (116);
receiving (204) data corresponding to signals output by the at least one sensing device upon activation by the subject during performance of a said physical task;
comparing (206) the received data with reference data, and
generating (208) an output based on the comparison representing an assessment of performance of the physical task by the subject.

18. A method according to claim 17, wherein a said physical task involves the subject activating the sensing devices (116A-116D) in a particular sequence/order.

19. A method according to claim 18, wherein the sensing devices are arranged in pattern (e.g. a zig-zag type arrangement) with a first subset (501, 503, 505) of the sensing devices being located to a left-hand (or right-hand) side of a notional line passing through the pattern and a second subset (502, 504, 506) of the sensing devices being located to a right-hand (or left-hand) side of the notional line.

20. A method according to claim 19, wherein the physical task involves the subject alternately activating a said sensing device in the first subset (501, 503, 505) and then activating a said sensing device in the second subset (502, 504, 506).

21. A method according to claim 19, including processing the data corresponding to the signals output by the sensing devices (116) to generate an output relating to performance of the physical task, the output being selected from a set including:
time taken by the subject to perform the physical task in its entirety;
time taken between the subject activating at least one sensing said device in the first subset (501, 503, 505) and at least one said sensing device in the second subset (502, 504, 506), or vice versa;
time taken by subject to progress between a first pair of said sensing devices in the sequence, a second pair of said sensing devices in the sequence, and so on;
approach speed of the subject to the sensing device, and/or
time spent by the subject in contact with at least some of the sensing devices in the sequence.

22. A method according to claim 17, wherein a said physical task includes the subject moving from one said sensing device (118A) to another said sensing device (118B).

23. A method according to claim 22, wherein the physical task includes a further activity in addition to moving from the sensing device (118A) to the other sensing device (118B).

24. A method according to claim 23, wherein the further activity involves a decision-making task and the method times/derives time taken by the subject in relation to the decision-making task.

25. A method according to claim 17, where in a said physical task involves the subject directly or indirectly applying physical force to a said sensing device (116), the sensing device outputting, in use, a signal corresponding to the physical force applied by the subject.

26. A method according to claim 17, wherein a said physical task includes the subject moving from one said sensing device to another said sensing device in a specific manner, e.g. running (in a backward/forward direction), jogging (in a backward/forward direction), or hopping on a specified leg.

27. A method according to claim 27, where, when the subject is hopping, then the method includes measuring times when the subject is hopping on each leg.

28. A method according to claim 26, wherein measurements taken or computed by the method include: time in flight whilst the subject is hopping; time on spent by the subject on the sensing devices; split times in flight and on the sensing devices; number of contacts per said sensing device; and/or differences between right and left leg/preseason/normal.

29-30. (canceled)
